(19) United States
(12) Patent Application Publication
(10) Pub. No.: US 2019/0183621 A1
(43) Pub. Date: Jun. 20, 2019

(54) METHOD OF APPLYING A DENTAL TREATMENT TO A PATIENT'S TEETH

(71) Applicant: 3M INNOVATIVE PROPERTIES COMPANY, St. Paul, MN (US)

(72) Inventors: Bruce R. Broyles, Oakdale, MN (US); Paul R. Klaiber, Mahtomedi, MN (US); Jennifer Post, St. Paul, MN (US)

(21) Appl. No.: 16/202,340

(22) Filed: Nov. 28, 2018

Related U.S. Application Data
(60) Provisional application No. 62/599,297, filed on Dec. 15, 2017.

(51) Int. Cl. A61C 19/06; A61Q 11/00; A61K 8/21; A61K 8/81

(52) U.S. Cl. CPC A61C 19/063; A61Q 11/00; A61K 2800/87; A61K 8/8147; A61K 8/21

(57) ABSTRACT

A method of applying a dental treatment composition to a patient's teeth, including selecting a dental treatment composition, applying the dental treatment composition to a surface of at least one tooth of the patient; and applying pressurized gas on the dental treatment composition to spread the dental treatment composition smoothly across the surface of the tooth.

### BACKGROUND

During a typical dental appointment, the dental hygienist will perform a dental prophylaxis procedure and may take dental X-rays for diagnostic procedures. A prophylaxis procedure is a professional cleaning that rids the patient's teeth of calcified plaque, called calculus or tartar, that builds up in hard-to-reach places. Also, the professional cleaning removes any stains by polishing the patient's teeth with a lightly abrasive paste.

After the prophylaxis procedure, a dental hygienist will evaluate the patient's risk of gum disease, tooth decay or root decay, and evaluate the patient's need for tooth restoration or tooth replacement. The dentist might recommend making a dental impression of one or both jaws to produce a replicate of the patient's teeth and oral tissue. The dental impression treatments involve using horseshoe-shaped trays and a soft gelatin-like impression material to create a cast of the patient's teeth to evaluate the patient's bite or make a mouth guard or bleaching trays for the patient.

After the dental exam, the dentist or hygienist might recommend certain preventative measures. One example of a preventative dental measure is to apply fluoride treatment to the teeth. Fluoride treatment involves the application of fluoride to a tooth surface with objective being the formation of fluorapatite and calcium fluoride.

There are two major in-office fluoride treatment methods currently in use. One treatment method uses a fluoride gel/foam in a tray. This method requires several grams of fluoride gel dispensed in a tray that is then placed into a patient's mouth onto the teeth. This tray is left in the mouth with the gel/foam in contact with the teeth for 1 to 4 minutes. This material requires the use of suction to pull the extra gel out of the mouth to avoid unnecessary high amounts of fluoride ingestion.

Another treatment method is a dental fluoride varnish. The varnish is painted on the teeth and remains in place for several hours to allow for the fluoride to be released from the composition. Typically, dentists use fluoride varnishes for in-office fluoride treatment. Such fluoride varnish treatments are more labor intensive than gel treatments. Compositions that are as simple to apply to teeth as varnishes and work in time periods as short as gel/foam formulations are desired.

The dentist might also perform various treatments by applying various dental compositions to the patient's teeth. Some examples of certain dental compositions include fluoride treatments, whitening treatments, color cosmetics, calculus softening agents, oral malodor treatments, biofilm disruption treatments, or combinations thereof. All these dental compositions are applied to the teeth by painting them onto the teeth with specialized brushes or other elaborate delivery systems, making them labor intensive.

### SUMMARY OF THE DISCLOSURE

The present disclosure provides a method of applying a dental treatment composition to a patient's teeth. In one embodiment, the method comprises: selecting a dental treatment composition; applying the dental treatment composition to a surface of at least one tooth of the patient; and applying pressurized gas on the dental treatment composition to spread the dental treatment composition smoothly across the surface of the tooth. Such methods are efficient ways of performing certain dental procedures in a dental office.

### BRIEF DESCRIPTION OF THE DRAWINGS

FIG. 1 illustrates a perspective view of one embodiment of a dental composition applicator;

FIG. 2 illustrates a perspective view of the dental composition applicator of FIG. 1 with the cap removed;

FIG. 3 illustrates applying the dental composition to a patient's teeth with the applicator of FIG. 2;

FIG. 4 illustrates the teeth after the dental composition has been initially applied;

FIG. 5 illustrates a pressurized gas device adjacent the teeth of FIG. 4;

FIG. 6 illustrates the pressurized air device of FIG. 5 applying pressurized gas on the dental composition on the teeth;

FIG. 7 illustrates the pressurized air device of FIG. 6 continuing to apply pressurized gas on the dental composition on the patient's teeth; and

FIG. 8 illustrates one embodiment after the method of the present invention of applying a dental composition on a patient's teeth is performed.

### DETAILED DESCRIPTION

In a dental office, there is always a need to reduce labor costs and to increase effectiveness of dental procedures. The present invention provides a quick, easy and consistent method of applying a dental composition on all areas of the tooth for certain dental procedures, lingually, facially and interproximally. Various dental care compositions include fluoride treatments, whitening treatments, color cosmetics, calculus softening agents, oral malodor treatments, biofilm disruption treatments, or combinations thereof. Preferably dental care compositions most suitable for the present invention are in the form of liquids. However, other dental care compositions are not suitable for use with the method of the present invention, specifically dental compositions of primers, adhesive or cement for applying a crown. This is because for those specific dental procedures it is necessary for a primer, adhesive or cement to stay in a precise location and not to be blown around the oral cavity, where it may spread to soft tissues, which may cause safety issues. Likewise, a dental restoration material for filling in a cavity or dental impression material are also not suitable dental compositions for use with the method of the present invention.

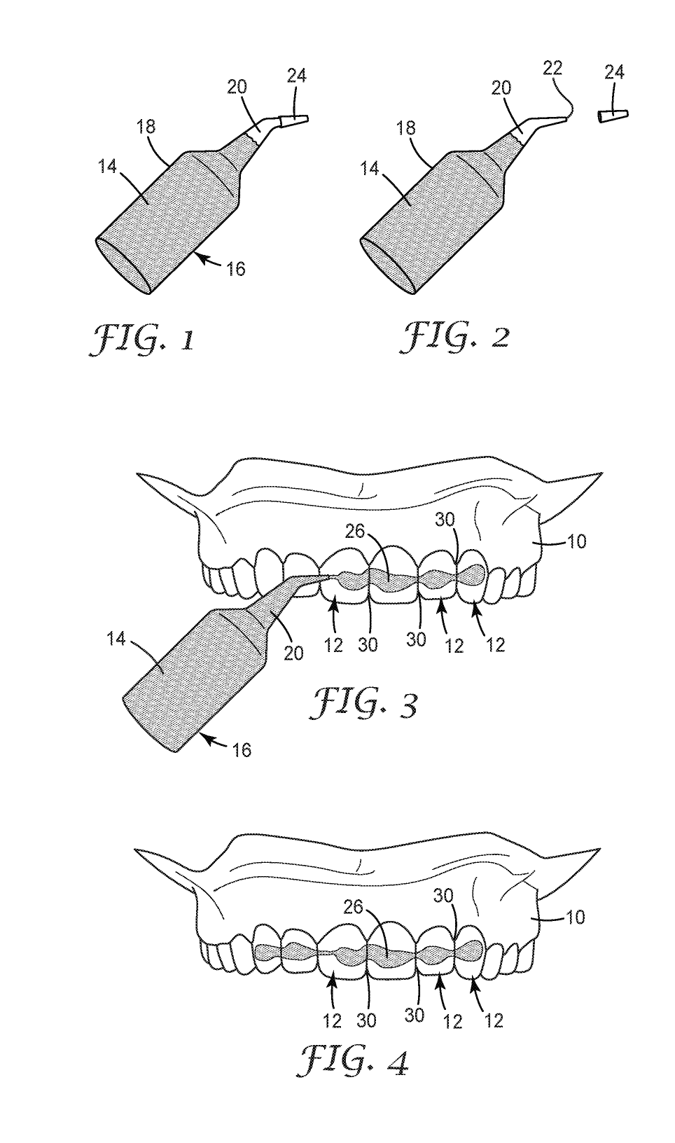

FIG. 1 illustrates one exemplary embodiment of an applicator 16 filled with a dental composition 14. For example, the dispenser 16 can be used to house and dispense various dental care compositions 14, such as fluoride treatments, whitening treatments, color cosmetics, calculus softening agents, oral malodor treatments, biofilm disruption treatments. The dental care composition 14 could be in the form of a gel, foam, varnish or liquid.

As illustrated, dental applicator 16 includes a body reservoir 18 for storing the dental composition 14 and a nozzle 20 in fluid communication with the reservoir 18. The nozzle's outlet 22 is covered by a cap 24. The cap 24 is removed prior to application to a patient's teeth 12. The dentist or hygienist then squeezes the reservoir 18 to force the dental composition 14 to exit through the nozzle 20 and out of the nozzle outlet 22. For example, the nozzle 20 and the nozzle outlet 22 can be made larger for a more viscous liquid, and smaller for a less viscous liquid. While the size of the nozzle 20 and the outlet 22 may be adjusted to accommodate liquids with varying viscosities, it may be desirable to select the viscosity of the liquid 14 such that it can be conveniently dispensed from the applicator 16. Suitable viscosities may range from about 0.5 Pa·s to about 500 Pa·s, from about 1 Pa·s to about 400 Pa·s, from about 5 Pa·s to about 300 Pa·s, or from about 10 Pa·s to about 200 Pa·s, at a shear rate of 1.0/s. However, the applicator 16 may be sized to accommodate the viscosity of the dental liquid 14 housed in the reservoir 18 and/or the intended use of the dental liquid 14.

The reservoir and nozzle 18, 20 may have any suitable shape. Although one embodiment is illustrated in the Figures, other shapes are also possible. The shape and orientation of the nozzle 20 and nozzle outlet 22 may also be designed such that they facilitate dispensing of the liquid 14 from the applicator 16.

The applicator 16 may be a single use applicator. However other applicators known in the art may be used, having either single or multiple uses.

The reservoir 18 or a portion of the reservoir 18 may be made of a transparent or translucent material so that the amount of liquid 14 in the reservoir 18 can be observed visually, as illustrated in the Figures. Alternatively, the reservoir 18 or a part of the reservoir 18 may be made of an opaque material that facilitates printing information on the wall of the reservoir 18, such as information about the contents, manufacturer, use, safety, expiration, etc.

Prior to application of the dental composition 14, a dental hygienist will typically perform a dental prophylaxis procedure and blow dry the teeth intended for treatment. The hygienist then removes the cap 24 from the applicator 16 to begin applying the dental composition 14.

FIG. 3 illustrates one embodiment of applying the dental treatment composition 14 to the surfaces of a patient's teeth 12 using the applicator 16. FIG. 3 illustrates the dental treatment composition 14 after it has been applied to the teeth. The dental composition 14 may only be applied to a single tooth 12 or may be applied to a plurality of teeth 12, depending on what is desired. Preferably, the dental composition 14 is applied in approximate uniform width and uniform thickness across the midline of a plurality of teeth 12. However, this is not essential for the present method. For instance, as illustrated in FIGS. 3 and 4, the dental composition 14 may be applied unevenly and in various widths and thicknesses across the teeth 12. As illustrated, a bead 26 of dental composition may be continuously applied to a plurality of teeth 12, preferably in the midline of the teeth. However, the dental composition 14 may be applied in multiple discontinuous portions intermittently or in dots to various teeth.

Adjacent teeth 12 have interproximal spaces 30 between them, and preferably the continuous bead of dental composition 14 is applied perpendicular to the direction of the interproximal spaces. However, the dental composition 14 could be applied in the direction of the interproximal spaces. The present method assists in evenly applying the dental composition 14 in such interproximal spaces 30, as discussed in more detail below.

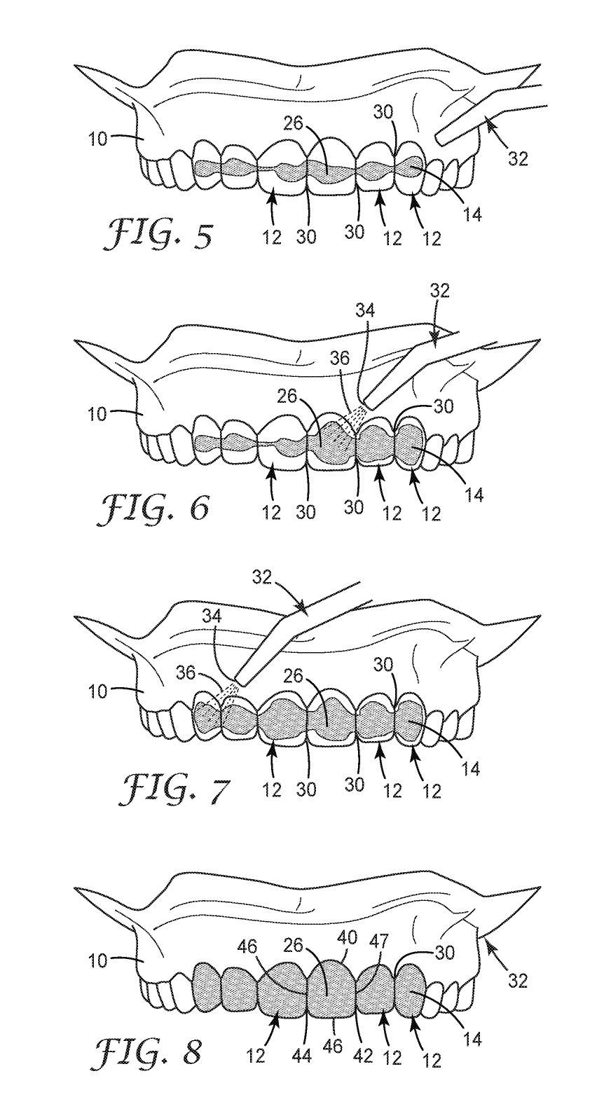

FIG. 5 illustrates a pressurized gas device 32 (partially shown) adjacent the teeth 12 and dental composition 14 on the teeth. FIGS. 6 and 7 illustrate the pressurized air device 32 blowing gas 36 on the dental composition 14 on the teeth smoothly across the surfaces of the teeth 12. The velocity of the pressurized gas 36 onto the teeth will be dependent on the viscosity of the dental composition 14. For example, the pressurized gas may be applied with a pressure greater than 10 psi for most typical dental treatments intended. The pressurized gas device 32 could be an air compressor that delivers at high pressure limits of 115 psi. One example of a suitable air compressor is an Osprey Compressor from RAMVAC (models OSP22, OSP13, OSP23, OSP24, OSP25, OSP28) commercially available from Dental EZ Integrated Solutions of Malvern, Pa., or Patterson Dental of St. Paul, Minn. Another example of an air compressor is AirStar Neo air compressors by AIR TECHNIQUES (Models such as AirStar 10 Neo, AirStar 21 Neo) commercially available from Patterson Dental of St. Paul, Minn. Alternatively, the pressurized gas device could be a typical air/water syringe found in most dental offices for delivering pressurized air 36. Optimum air pressure with a typical dental air/water syringe is 40-80 psi. Such syringes are used to dry the teeth or to blow scaled calculus off the teeth. One example of such a syringe is a Johnson-Promident 3-Way Air/Water Syringe commercially available from Patterson Dental Supply Inc., Patterson Item #: 404-1893. Regardless, the gas is blown by some pressurized gas source, and could be air or some other inert gas or gas mixture. For example, the gas could be nitrogen, helium, argon, carbon dioxide, or nitrous oxide. Also, the pressurized gas device 32 could be part of a permanently installed “in-house” pressurized air/gas system or a hand held, self-contained canister.

Using a pressurized air device 32 eliminates the need for the dentist or the hygienist to manually paint on the dental composition 14 with a brush or a sponge, thus saving valuable time. Moreover, the pressurized air device ensures an even, consistent thickness of the dental composition 14 over the entire surface of the tooth, including in those hard to reach interproximal spaces 30 between adjacent teeth 12. In addition, blowing pressurized air actively removes some of the volatile solvent, for example water in the case of aqueous compositions, from the dental composition 14 during the application process, and thus thickens the composition on the surface of the tooth. This thickening may enhance the dental treatment effectiveness of the composition 14 compared to prior art methods.

FIG. 8 illustrates one embodiment of the patient's teeth 12 after the present method. The dental composition 14 is now spread across both the lingual surfaces of the selected teeth 12 and between the interproximal spaces 30 between adjacent teeth 12. Preferably the layer of the dental composition 14 extends in a thin layer from the gingival portion 40 of the tooth 12 near gums 10 to the incisal surface 46 of the tooth 12, and from the mesial surface 42 of the tooth 12 to the distal surface 44 of the tooth, as well as the interproximal surfaces of the tooth 12. This ensures all portions of the tooth receives the dental treatment desired by the dental composition so applied.

In one embodiment, the dental treatment composition 14 may be an aqueous oral care fluoride treatment composition. Fluoride therapy has been proven to have a beneficial effect on the prevention of dental caries (tooth decay). Dental caries caused by acid-producing bacteria that collect around the teeth and gingivae (gums) in a sticky film called plaque. Dentists have used in-office fluoride treatments for decades to help protect the oral health of children and adults, especially patients who may be at a higher risk of developing caries. Fluoride may be in the form of a solution, gel, foam or varnish. In typical prior art methods, it is applied with a cotton swab or brush, or it is used as a rinse. After the treatment, you may be asked not to rinse, eat or drink for at least 30 minutes to allow the teeth to absorb the fluoride and help repair the microscopic carious areas. In other typical prior art methods, a fluoride gel is placed in a tray that is held in the mouth for several minutes. The fluoride gel often slumps out of the tray into the back of the mouth and throat. This often causes the patient to gag making it an undesirable dental procedure especially with younger patients. To prevent swallowing excessive quantities of the fluoride gel, the dentist or hygienist uses an evacuator in the mouth, along with the trays, during the entire treatment period, which last up to five minutes.

In contrast, the present inventive method of air spreading a fluoride treatment ensures that the fluoride containing composition is consistently and evenly applied over the entire surface of the tooth or teeth 12 to be treated easily and quickly, including in between the teeth in the interproximal regions 30. The interproximal regions 30 are often considered prone to caries. The present method also assists in removing water or other liquids from the composition after it is blown onto the tooth to make the fluoride composition more retentive to the tooth surface, thereby increasing treatment time to allow the patient's teeth to absorb the fluoride. As a result, the present inventive method may further reduce the incidence of dental caries in a patient because it may assist in increasing the efficiency of the fluoride uptake on the treated teeth. To deposit fluoride on a tooth surface, sodium fluoride needs to form a solution in water to be ionized. The fluoride ion reacts with calcium ion and hydroxyapatite to form calcium fluoride and fluorapatite on the tooth surface. When the pressurized air spreads the fluoride over the tooth surface, the deposition reaction starts immediately, so the treatment can be very short. Since the reaction is also controlled by the diffusion of ions, the longer the time of contact with the fluoride ion solution leads to better fluoride uptake. For dental fluoride varnishes sodium, sodium fluoride is dispersed in an organic solvent composition, sodium fluoride releases from the solid composition in saliva to form fluoride ion, which is diluted by saliva. The reaction is controlled by the release rate and concentration, but dental fluoride varnish can provide longer release of fluoride ion with lower concertation, fluoride deposits on the tooth surface. The present inventive method may provide better fluoride deposition on a tooth compared with prior art methods described in the Background section above.

One example of aqueous fluoride treatments suitable for use in the present inventive method are taught in “Aqueous Oral Care Fluoride Treatment Compositions, and Methods,” U.S. Patent Application No. 62/556,095, filed on Sep. 8, 2017, which is hereby incorporated by reference. Another example of aqueous fluoride treatments suitable for use in the present inventive method are taught in “Aqueous Oral Care Fluoride Treatment Compositions, and Methods,” U.S. Patent Application No. 62/565,345, filed on Sep. 29, 2017, which is hereby incorporated by reference.

The present inventive method of applying dental treatment compositions may use one of the two following exemplary dental treatment compositions.

The first exemplary dental treatment composition includes at least 60 wt-%, at least 70 wt-%, or at least 80 wt-%, water, based on the total weight of the aqueous composition. In certain embodiments, a treatment composition includes up to 96 wt-%, or up to 90 wt-%, water, based on the total weight of the aqueous composition.

The first exemplary dental treatment composition may include a small amount of organic solvent (e.g., (C1-C4) alcohols such as ethanol), preferably they are free of organic solvents that function as liquid carriers (as opposed to organic solvents that are used as carriers/solvents for flavorants or sweeteners). For example, certain additives may be provided as a solution or dispersion in an organic solvent as a liquid carrier. If there is any organic solvent (that functions as a liquid carrier) present in aqueous oral care fluoride treatment compositions of the present disclosure, it is present in an amount of less than 5 wt-%, based on the total weight of the aqueous composition.

The first exemplary dental treatment composition includes a pharmaceutically acceptable buffer. The type and amount of such buffer is selected to provide a treatment composition with a pH of at least 6, or at least 6.5. In certain embodiments, the type and amount of such buffer is selected to provide a treatment composition with a pH of up to 8, up to 7.5, or up to 7. In certain embodiments, the type and amount of such buffer is selected to provide a treatment composition with a pH of 6.5 to 7.5, or a pH of 7.0, particularly for methods of reducing the incidence of dental caries, according to the Monograph entitled “Topical Fluoride Preparations for Reducing Incidence of Dental Caries,” Federal Register, Vol. 39, No. 94, May 14, 1974.

The first exemplary dental treatment composition includes a water-soluble polymer; however, typically the compositions are not gels. In general, gels include polymer chains that are chemically crosslinked and extended or swollen by solvents (e.g., water). Thus, typically, compositions of the present disclosure are not crosslinked, although they may include polymer chains that have non-chemically bonded linkages that are extended in the presence of a solvent (e.g., water).

The first exemplary dental treatment composition includes a water-soluble cationic N-containing polymer. Typically, herein a water-soluble cationic N-containing polymer has a solubility in water at room temperature of greater than 1 gram, greater than 2 grams, greater than 3 grams, greater than 4 grams, or greater than 5 grams, per 100 grams of water.

In certain embodiments, the first exemplary dental treatment composition includes a water-soluble cationic N-containing polymer in an amount of at least 0.5 wt-%, or at least 0.6 wt-%, or at least 0.9 wt-%, based on the total weight of the aqueous composition. In certain embodiments, an aqueous oral care fluoride treatment composition of the present disclosure includes a water-soluble cationic N-containing polymer in an amount of up to 4.8 wt-%, or up to 4.0 wt-%, based on the total weight of the aqueous composition.

In certain embodiments, suitable water-soluble cationic N-containing polymers may not be cationic, may not be water soluble, or may not be either, prior to incorporation into a treatment composition. Such polymers are referred to as “polymer precursors” herein. For example, a water-insoluble basic polymer (e.g., EUDRAGIT E100 poly(butyl methacrylate-co-(2-dimethylaminoethyl) methacrylate-co-methyl methacrylate copolymer) may become cationic upon contact with an acidic aqueous solution. Typically, upon forming a cationic polymer, such polymers have a water solubility at room temperature of greater than 1 gram per 100 grams of water.

Examples of classes of suitable water-soluble cationic N-containing polymers or polymer precursors, used in the first exemplary dental treatment composition, includes include the following: (meth)acrylate copolymers containing dimethylaminoethyl side groups, such as those copolymers derived from only (meth)acrylate monomers or those copolymers derived from (meth)acrylate monomers and other monomers such as vinyl monomers; polyethylenimines; and cationic-modified polysaccharides. These polymers may include nitrogen in the backbone and/or side groups. Various combinations of such polymers can be used if desired.

The first exemplary dental treatment composition includes sodium fluoride as the active agent. In certain embodiments, a treatment composition includes at least 1.0 wt-%, based on the total weight of the aqueous composition. In certain embodiments, a treatment composition includes up to 2.5 wt-%, based on the total weight of the aqueous composition.

The second exemplary dental treatment composition includes at least 60 wt-%, at least 70 wt-%, or at least 80 wt-%, water, based on the total weight of the aqueous composition. In certain embodiments, a treatment composition includes up to 96 wt-%, or up to 90 wt-%, water, based on the total weight of the aqueous composition.

The second exemplary dental treatment composition may include a small amount of organic solvent (e.g., (C1-C4) alcohols such as ethanol), preferably they are free of organic solvents that function as liquid carriers (as opposed to organic solvents that are used as carriers/solvents for flavorants or sweeteners). For example, certain additives may be provided as a solution or dispersion in an organic solvent as a liquid carrier. If there is any organic solvent (that functions as a liquid carrier) present in aqueous oral care fluoride treatment compositions of the present disclosure, it is present in an amount of less than 5 wt-%, based on the total weight of the aqueous composition.

The second exemplary dental treatment composition includes a pharmaceutically acceptable buffer. The type and amount of such buffer is selected to provide a treatment composition with a pH of at least 6, or at least 6.5. In certain embodiments, the type and amount of such buffer is selected to provide a treatment composition with a pH of up to 8, up to 7.5, or up to 7. In certain embodiments, the type and amount of such buffer is selected to provide a treatment composition with a pH of 6.5 to 7.5, or a pH of 7.0, particularly for methods of reducing the incidence of dental caries, according to the Monograph entitled “Topical Fluoride Preparations for Reducing Incidence of Dental Caries,” Federal Register, Vol. 39, No. 94, May 14, 1974.

The second exemplary dental treatment composition includes a crosslinked poly acid. The crosslinked poly acid can be a homopolymer or a copolymer of one or more monomers having one or more carboxylic acid groups.

Examples of monomers having one or more carboxylic acid groups include acrylic acid, maleic acid, maleic anhydride, itaconic acid, vinyl ether, alkyl acrylates, and saccharides. Various combinations of such monomers may be used if desired. In certain embodiments, the crosslinked poly acid includes a homopolymer or a copolymer of acrylic acid, maleic acid, itaconic acid, (C10-C30)alkyl acrylate, and a combination thereof. In certain embodiments, the crosslinked poly acid includes a crosslinked polyacrylic acid homopolymer.

Examples of crosslinkers include allyl sucrose, allyl pentaerythritol, glycerol, diglycerol, polyglycerol, polyalkenyl alcohols, or divinyl glycol. Various combinations of crosslinkers may be used if desired. In certain embodiments, the polyacrylic acid is crosslinked with allyl sucrose or allyl pentaerythritol.

The second exemplary dental treatment composition may include poly acids such as linear polyacrylic acids, acrylic acid copolymers, maleic acid/maleic anhydride copolymers, itaconic acid copolymers, vinyl ether and maleic anhydride copolymers such as that available under the tradename GANTREZ (from Ashland), high molecular weight crosslinked polyacrylic acid homopolymers and copolymers such as that available under the tradename CARBOPOL (from Lubrizol Life Sciences), and carboxylic acid modified cellulose (CMC) such as that available under the tradename KLUCEL (from Ashland). These homopolymers and copolymers have the same types of carboxylic acid side groups that can be neutralized with some alkaline chemicals to form an aqueous buffer system. These polymers are water soluble or dispersible or capable of forming a hydrogel with water solution.

The second exemplary dental treatment composition includes suitable multivalent cation salts such as those that include a +2 multivalent cation, a +3 multivalent cation, or a combination thereof. In certain embodiments, the multivalent cation salts include a +3 multivalent cation. Various combinations of such salts may be used if desired.

Examples of suitable multivalent cations in the salts include those selected from Ca, Mg, Ba, Mn, Fe, Zn, Al, Cu, and a combination thereof. In certain embodiments, the multivalent cation is selected from Ca, Zn, Al, and a combination thereof. In certain embodiments, the multivalent cation includes Al and Ca.

Examples of suitable counterions in the multivalent cation salts include those selected from chloride, nitrate, gluconate, lactate, acetate, and sulfate. Various combinations of counterions may be used if desired. Also, the salts may also include hydrates thereof.

Compositions of the second exemplary dental treatment composition include at least 0.025 wt-%, at least 0.05 wt-%, or at least 0.1 wt-%, of a multivalent cation salt, based on the total weight of the aqueous composition. Compositions of the present disclosure include up to 1.75 wt-%, up to 1.5 wt-%, or up to 1.0 wt-%, of a multivalent cation salt, based on the total weight of the aqueous composition.

The second exemplary dental treatment composition includes sodium fluoride as the active agent. In certain embodiments, a treatment composition includes at least 1.0 wt-%, based on the total weight of the aqueous composition. In certain embodiments, a treatment composition includes up to 2.5 wt-%, based on the total weight of the aqueous composition.

### Exemplary Embodiments

Embodiment 1 is a method of applying a dental treatment composition to a patient's teeth, comprising: selecting a dental treatment composition; applying the dental treatment composition to a surface of at least one tooth of the patient; and applying pressurized gas on the dental treatment composition to spread the dental treatment composition smoothly across the surface of the tooth.

Embodiment 2 is the method of embodiment 1, wherein the applying step comprises applying the dental treatment composition to the surfaces of a plurality of teeth.

Embodiment 3 is the method of embodiment 2, wherein the applying step comprises applying the dental treatment composition in approximate uniform width and uniform thickness, across a midline of a plurality of teeth.

Embodiment 4 is the method of embodiment 2, wherein the applying step comprises applying the dental treatment composition to the surfaces of a plurality of teeth in multiple intermittent portions.

Embodiment 5 is the method of embodiment 2, wherein the applying step comprises applying the dental treatment composition in a continuous linear bead across adjacent surfaces of the plurality of teeth.

Embodiment 6 is the method of embodiment 1, wherein the dental treatment composition comprises a liquid.

Embodiment 7 is the method of embodiment 6, wherein the liquid dental treatment composition is a gel, foam or varnish.

Embodiment 8 is the method of embodiment 1, wherein the dental treatment composition has a viscosity greater than 0.5 Pascal seconds at a shear rate of 1.0/second.

Embodiment 9 is the method of embodiment 1, wherein the dental treatment composition has a viscosity less than 500 Pascal seconds at a shear rate of 1.0/second.

Embodiment 10 is the method of embodiment 1, further comprising: providing the liquid dental treatment composition in an applicator; and dispensing the dental treatment composition from the applicator onto the surface of the at least one tooth.

Embodiment 11 is the method of embodiment 1, wherein the dental treatment composition comprises compositions to provide fluoride treatments, whitening treatments, color cosmetics, calculus softening agents, oral malodor treatments, biofilm disruption treatments, or combinations thereof.

Embodiment 12 is the method of embodiment 1, wherein the dental treatment composition comprises an aqueous oral care fluoride treatment composition.

Embodiment 13 is the method of embodiment 12, wherein the applying pressurized gas step accelerates the evaporation of water from the aqueous fluoride treatment and increases the fluoride concentration of the aqueous oral care fluoride treatment composition on the surface of the tooth.

Embodiment 14 is the method of embodiment 12, wherein the aqueous oral care fluoride treatment composition comprises: 0.5 wt-% to 4.8 wt-% of a water-soluble cationic N-containing polymer; a pharmaceutically acceptable buffer; 1.0 wt-% to 2.5 wt-% of sodium fluoride; and at least 60 wt-% water; wherein the weight percentages are based on the total weight of the aqueous composition.

Embodiment 15 is the method of embodiment 12, wherein the aqueous oral care fluoride treatment composition comprises: 0.1 wt-% to 3.0 wt-% of a crosslinked poly acid having carboxylic acid side groups; a pharmaceutically acceptable buffer; 1.0 wt-% to 2.5 wt-% of sodium fluoride; and 0.025 wt-% to 1.75 wt-% of a multivalent cation salt; and at least 60 wt-% water; wherein the weight percentages are based on the total weight of the aqueous composition.

Embodiment 16 is the method of embodiment 1, wherein the dental treatment composition comprises non-aqueous oral care fluoride treatment composition.

Embodiment 17 is the method of embodiment 16, wherein the applying pressurized gas step accelerates the evaporation of non-water solvents and increases the fluoride concentration of the aqueous oral care fluoride treatment composition on the surface of the tooth.

Embodiment 18 is the method of embodiment 1, wherein the pressurized gas is air or an inert gas.

Embodiment 19 is the method of embodiment 1, wherein the gas is nitrogen, helium, argon, or carbon dioxide, or nitrous oxide.

Embodiment 20 is the method of embodiment 1, wherein the pressurized gas is applied with a pressure greater than 10 psi.

Embodiment 21 is the method of embodiment 1, wherein prior to the applying step, a dental prophylaxis procedure is performed on the at least one surface of at least one tooth of the patient.

Embodiment 22 is the method of embodiment 1, wherein the applying the pressurized gas step includes spreading the dental treatment composition into the interproximal areas next to the tooth.

Embodiment 23 is the method of embodiment 1, wherein the applying pressurized gas step includes removing some water from the dental composition which then thickens the dental treatment composition on the surface of the tooth.

Embodiment 24 is the method of embodiment 1, wherein the dental treatment composition is not a dental adhesive, cement, dental restoration material, or dental impression material.

Embodiment 25 is the method of reducing the incidence of dental caries in a patient in need thereof, the method comprising applying an aqueous oral care fluoride treatment composition of embodiment 13 to the patient's tooth surface by the method of embodiment 1.

Embodiment 26 is a method of reducing the incidence of dental caries in a patient in need thereof, the method comprising applying an aqueous oral care fluoride treatment composition of embodiment 14 to the patient's tooth surface by the method of embodiment 1.

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

What is claimed is:

1. A method of applying a dental treatment composition to a patient's teeth, comprising:
selecting a dental treatment composition;
applying the dental treatment composition to a surface of at least one tooth of the patient; and
applying pressurized gas on the dental treatment composition to spread the dental treatment composition smoothly across the surface of the tooth.

2. The method of claim 1, wherein the applying step comprises applying the dental treatment composition to the surfaces of a plurality of teeth.

3. The method of claim 1, wherein the applying step comprises applying the dental treatment composition in approximate uniform width and uniform thickness, across a midline of a plurality of teeth.

4. The method of claim 1, wherein the applying step comprises applying the dental treatment composition to the surfaces of a plurality of teeth in multiple intermittent portions.

5. The method of claim 1, wherein the applying step comprises applying the dental treatment composition in a continuous linear bead across adjacent surfaces of the plurality of teeth.

6. The method of claim 1, wherein the dental treatment composition comprises a liquid.

7. The method of claim 6, wherein the liquid dental treatment composition is a gel, foam or varnish.

8. The method of claim 1, wherein the dental treatment composition has a viscosity greater than 0.5 Pascal seconds at a shear rate of 1.0/second.

9. The method of claim 1, wherein the dental treatment composition has a viscosity less than 500 Pascal seconds at a shear rate of 1.0/second.

10. The method of claim 1, further comprising:
providing the dental treatment composition in an applicator; and
dispensing the dental treatment composition from the applicator onto the surface of the at least one tooth.

11. The method of claim 1, wherein the dental treatment composition comprises compositions to provide fluoride treatments, whitening treatments, color cosmetics, calculus softening agents, oral malodor treatments, biofilm disruption treatments, or combinations thereof.

12. The method of claim 1, wherein the dental treatment composition comprises an aqueous oral care fluoride treatment composition.

13. The method of claim 12, wherein the applying pressurized gas step accelerates the evaporation of water from the aqueous fluoride treatment and increases the fluoride concentration of the aqueous oral care fluoride treatment composition on the surface of the tooth.

14. The method of claim 12, wherein the aqueous oral care fluoride treatment composition comprises:
0.5 wt-% to 4.8 wt-% of a water-soluble cationic N-containing polymer;
a pharmaceutically acceptable buffer;
1.0 wt-% to 2.5 wt-% of sodium fluoride; and
at least 60 wt-% water;
wherein the weight percentages are based on the total weight of the aqueous composition.

15. The method of claim 12, wherein the aqueous oral care fluoride treatment composition comprises:
0.1 wt-% to 3.0 wt-% of a crosslinked poly acid having carboxylic acid side groups;
a pharmaceutically acceptable buffer;
1.0 wt-% to 2.5 wt-% of sodium fluoride; and
0.025 wt-% to 1.75 wt-% of a multivalent cation salt; and
at least 60 wt-% water;
wherein the weight percentages are based on the total weight of the aqueous composition.

16. The method of claim 1, wherein the dental treatment composition comprises non-aqueous oral care fluoride treatment composition.

17. The method of claim 16, wherein the applying pressurized gas step accelerates the evaporation of non-water solvents and increases the fluoride concentration of the aqueous oral care fluoride treatment composition on the surface of the tooth.

18. The method of claim 1, wherein the pressurized gas is air or an inert gas.

19. The method of claim 1, wherein the gas is nitrogen, helium, argon, or carbon dioxide, or nitrous oxide.

20. The method of claim 1, wherein the pressurized gas is applied with a pressure greater than 10 psi.

21. The method of claim 1, wherein prior to the applying step, a dental prophylaxis procedure is performed on the at least one surface of at least one tooth of the patient.

22. The method of claim 1, wherein the applying the pressurized gas step includes spreading the dental treatment composition into the interproximal areas next to the tooth.

23. The method of claim 1, wherein the applying pressurized gas step includes removing some water from the dental composition which then thickens the dental treatment composition on the surface of the tooth.

24. The method of claim 1, wherein the dental treatment composition is not a dental adhesive, cement, dental restoration material, or dental impression material.

25. A method of reducing the incidence of dental caries in a patient in need thereof, the method comprising applying an aqueous oral care fluoride treatment composition of claim 13 to the patient's tooth surface by:
applying the aqueous oral care fluoride treatment composition to a surface of at least one tooth of the patient; and
applying pressurized gas on the aqueous oral care fluoride treatment composition to spread the dental treatment composition smoothly across the surface of the tooth.

26. A method of reducing the incidence of dental caries in a patient in need thereof, the method comprising applying an aqueous oral care fluoride treatment composition of claim 14 to the patient's tooth surface by:
applying the aqueous oral care fluoride treatment composition to a surface of at least one tooth of the patient; and
applying pressurized gas on the aqueous oral care fluoride treatment composition to spread the dental treatment composition smoothly across the surface of the tooth.
